(19) United States
(12) Patent Application Publication
(10) Pub. No.: US 2013/0344587 A1
(43) Pub. Date: Dec. 26, 2013

(54) FOAM REMOVAL DEVICE IN AUTOMATIC CELL HANDLING ROBOT

(71) Applicants: Koichi Nakayama, Saga (JP); Takeshi Shimoto, Fukuoka (JP)

(72) Inventors: Koichi Nakayama, Saga (JP); Takeshi Shimoto, Fukuoka (JP)

(73) Assignee: SAGA UNIVERSITY, Saga-shi, Saga (JP)

(21) Appl. No.: 13/984,155

(22) PCT Filed: Feb. 15, 2012
(86) PCT No.: PCT/JP2012/053460

§ 371 Date: Sep. 12, 2013

(30) Foreign Application Priority Data
Feb. 15, 2011 (JP) 2011-030043

(51) Int. Cl. C12M 1/00

(52) U.S. Cl. CPC C12M 21/00
USPC 435/297.1; 435/289.1

(57) ABSTRACT

A system for culturing a cell mass comprising:
handling means for handling the cell mass;
a first container for reserving the cell mass supplied from the handling means, the first container having micropores that allow communication of a culture fluid with the external area;
a second container for accommodating the first container, where a culture fluid fills around the first container in an excessive amount as compared to the amount of the culture fluid in the first container; and
liquid removal means for removing a droplet or foam remaining in the handling means after supplying the cell mass into the first container.

### TECHNICAL FIELD

The present invention relates to a cell culture system, and more specifically to a foam removal device in an automatic cell handling robot for culturing cell masses in a three-dimensional form by employing a droplet or foam removal unit.

### BACKGROUND ART

Recently, instrumentation techniques are gradually developing in the biotechnology field. Since instrumentation of conventional manual labors is expected to result effects such as highly accurate and highly efficient operations and reduction in contamination risk, studies have been continued. For example, Patent Document 1 describes a system for treating a biological sample as represented by a DNA microarray, and shows a system for efficiently treating a biological sample. Furthermore, Patent Document 2 describes a treatment process for reducing non-specific adsorption that occurs in a microplate, a microtube, a pipette tip or the like used with a lab-on-a-chip or the like, so as to solve the problems caused in conventional devices. Moreover, Patent Document 3 describes a blood-collecting device that allows collection of a prescribed amount of blood without introducing bubbles and a pipette attached to and used with this blood-collecting device.

On the other hand, research in the field of regenerative medicine is rapidly making progress. In regenerative medicine, for example, artificially cultured cells are used instead of disrupted cells at an affected site as regeneration therapy for the affected site. As a method for culturing said cells, 2D culture in which the cells are cultured in Schale, or petri dish, is conventionally well known.

Meanwhile, in the case of applying the cultured cells to the affected site, it is difficult to retain the cells in a useful form at the affected site, and for this reason, in some cases, an intended therapeutic effect cannot be achieved. In this respect, in order to achieve the therapeutic effect in a more certain way, it was considered to apply the cells in an amount adequate for the treatment to the affected site. In order to culture the cells in an amount that is adequate for the treatment, however, the cultivation requires a long period of time, for example, of several weeks. Additionally, the property of the cultured cells may change during such a long period of cell cultivation, which sometimes results in shortage of the cell amount required for transplantation. Hence, it was considered to sterically culture the cells so as to acquire the cells in a form of a three-dimensional construct in an amount adequate for treating the affected site (Patent Document 4). Manual formation of the three-dimensional cell construct, however, has many issues regarding efficiency and also a possibility of man-caused operational mistakes such as contamination.

### PRIOR ART DOCUMENTS

### Patent Documents

[Patent Document 1] Japanese Unexamined Patent Application Publication No. 2004-163408

[Patent Document 2] Japanese Unexamined Patent Application Publication No. 2010-202823

[Patent Document 3] Japanese Unexamined Patent Application Publication No. 2005-017281

[Patent Document 4] Japanese Unexamined Patent Application Publication No. 2004-357694

### SUMMARY OF THE INVENTION

### Problem to be Solved by the Invention

Regarding the above-described circumstances, the present invention has an objective of providing a culture system that is convenient and capable of preparing a three-dimensional cell construct by culturing cell masses in an inexpensive and safe manner while preventing occurrence of contamination.

### Means for Solving the Problem

In order to accomplish the above-described objective, the culture system of the present invention is characterized by comprising:
handling means for handling a cell mass;
a first container for reserving the cell mass supplied from the handling means, the first container having micropores that allow communication of a culture fluid with the external area;
a second container for accommodating the first container, where a culture fluid that fills around the first container is in an excessive amount as compared to the amount of the culture fluid in the first container; and
liquid removal means for removing a droplet or foam remaining in the handling means after supplying the cell mass into the first container.

Preferably, this handling means comprises a nozzle capable of drawing in and discharging a liquid (a culture fluid containing the cell mass). Additionally, a disposable tip may be attached to this nozzle.

Moreover, the liquid removal means is characterized by comprising a drainer unit for removing a droplet or foam, which is caused upon the above-described drawing or discharge, from the end of the nozzle when the end of the nozzle approaches the drainer unit.

Moreover, in the culture system of the present invention, the first container is a mold for molding a three-dimensional cell construct by culturing the reserved cell masses, wherein a member (filter) having micropores is provided at the bottom of the mold.

In addition, the culture system of the present invention has a funnel that opens outward at the opening of the first container.

Furthermore, the present invention is a drainer unit for removing a droplet or foam, which is caused upon drawing or discharge, near the end of the nozzle provided on the handling means that is capable of drawing and discharging a culture fluid, wherein the drainer unit is provided with an aperture having a size that allows the nozzle to pass up and down therethrough.

The above-described unit removes the droplet or the foam from the end of the nozzle by making the droplet or the foam to make contact with the unit in the vicinity of the aperture. Preferably, this unit is placed at the opening of the culture container.

### Effect of the Invention

When cells are handled using a cell culture apparatus, a tool that makes contact with the culture fluid is the nozzle of the apparatus or the tip provided at the end thereof. According to the present invention, removal of a droplet or foam caused at the nozzle or the tip can prevent bubble generation that hinders preparation of the cell masses. Thus, according to the present invention, a three-dimensional cell construct can be prepared by conveniently culturing cell masses while preventing contamination.

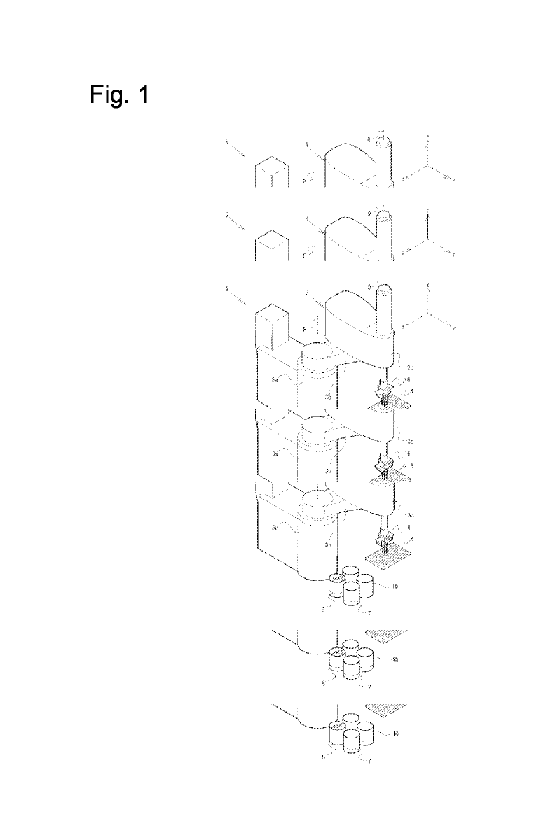

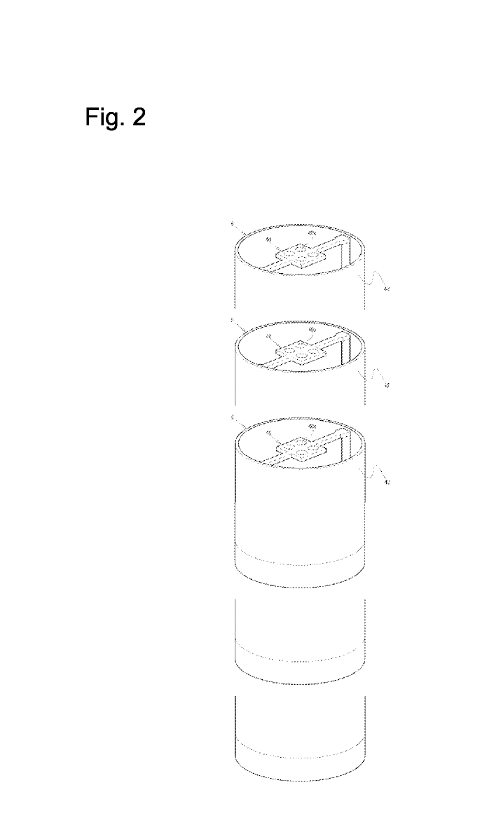

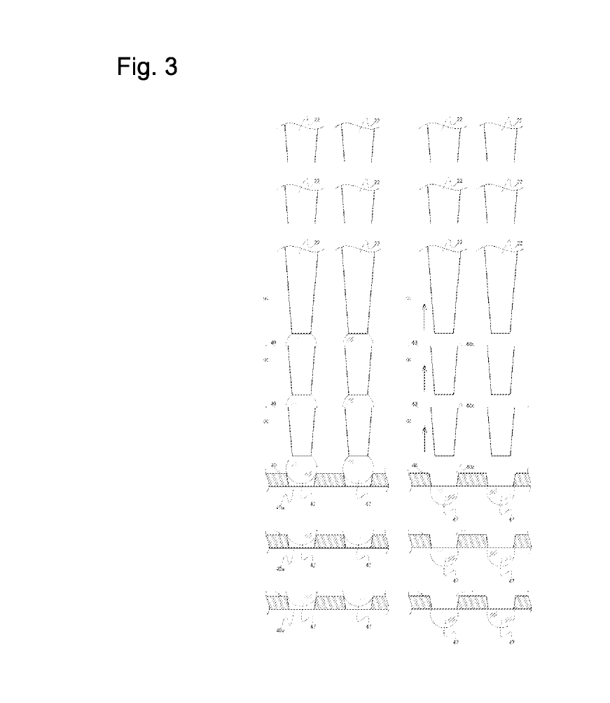

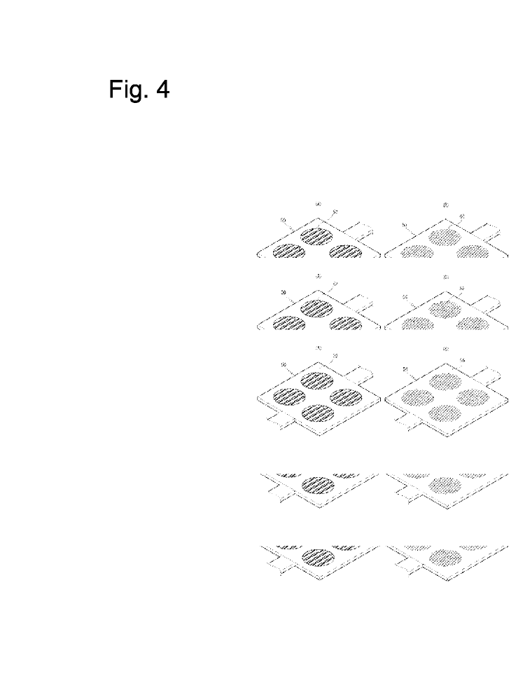

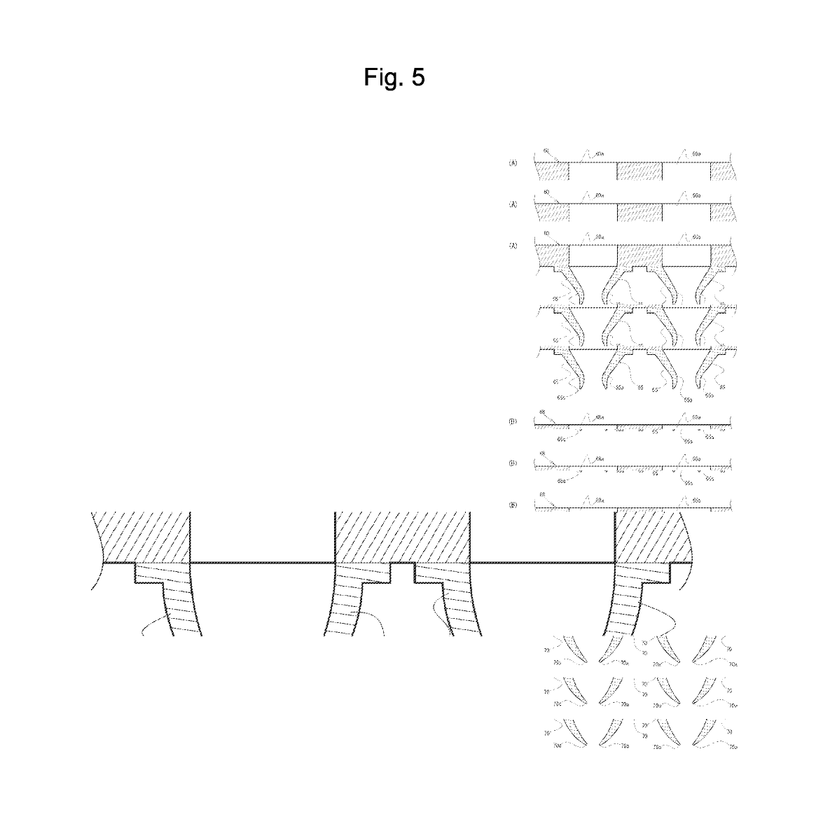

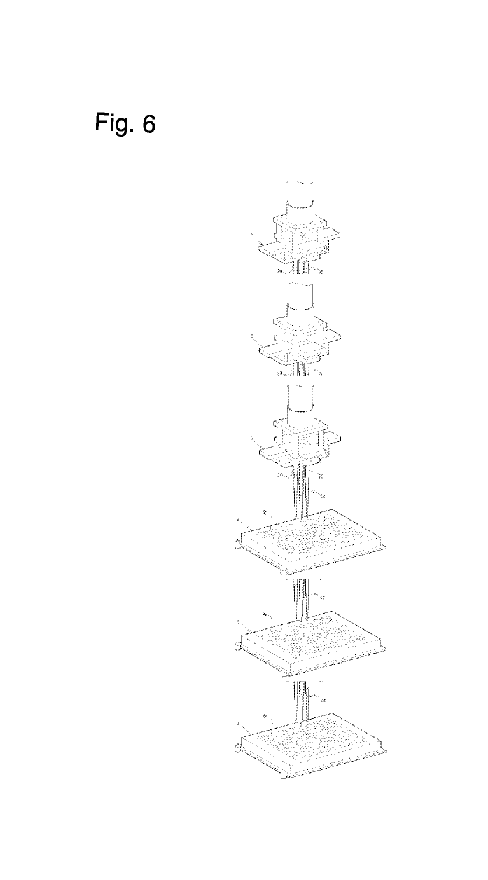

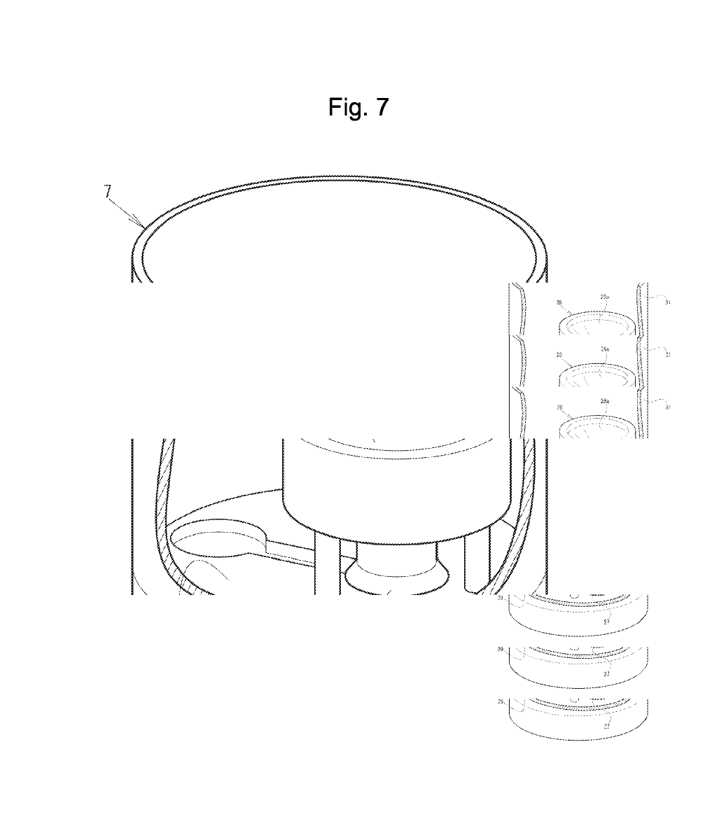

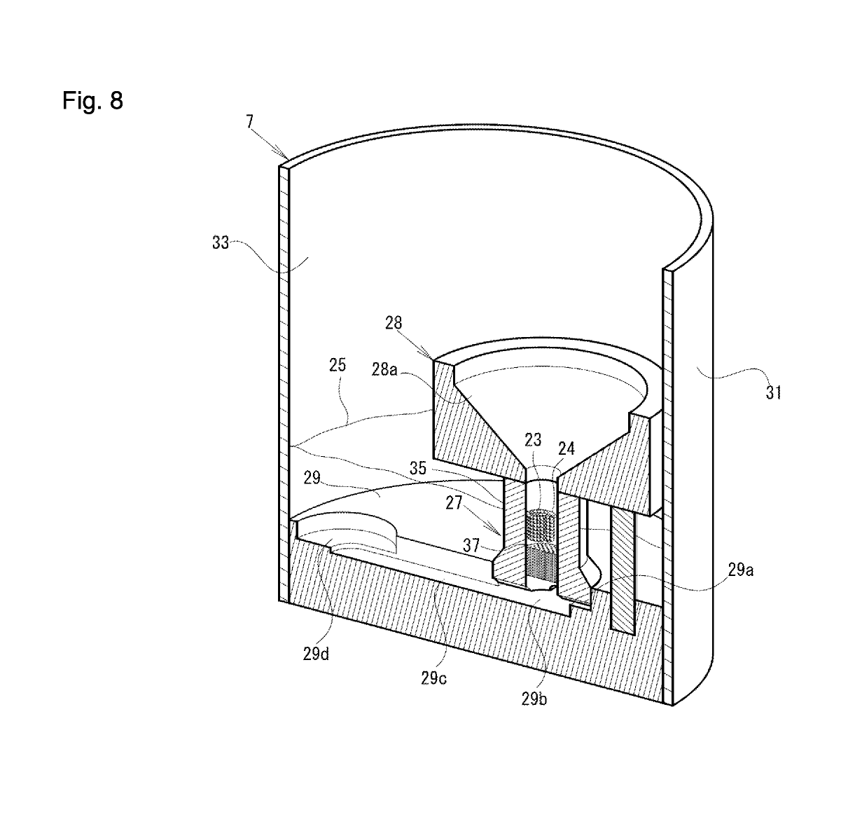

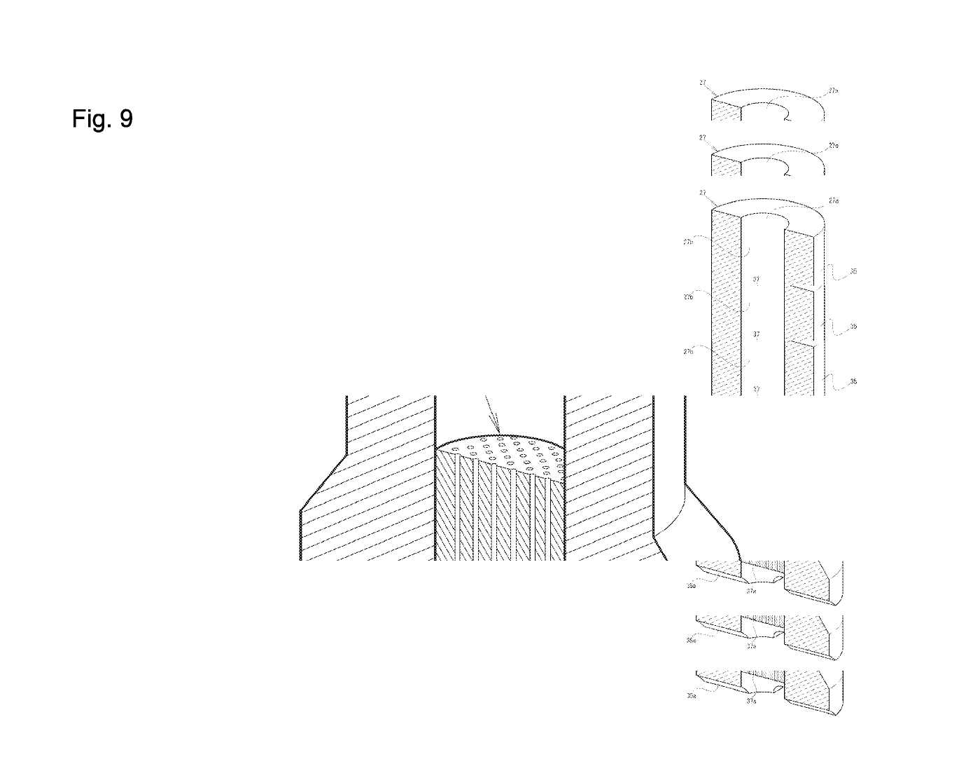

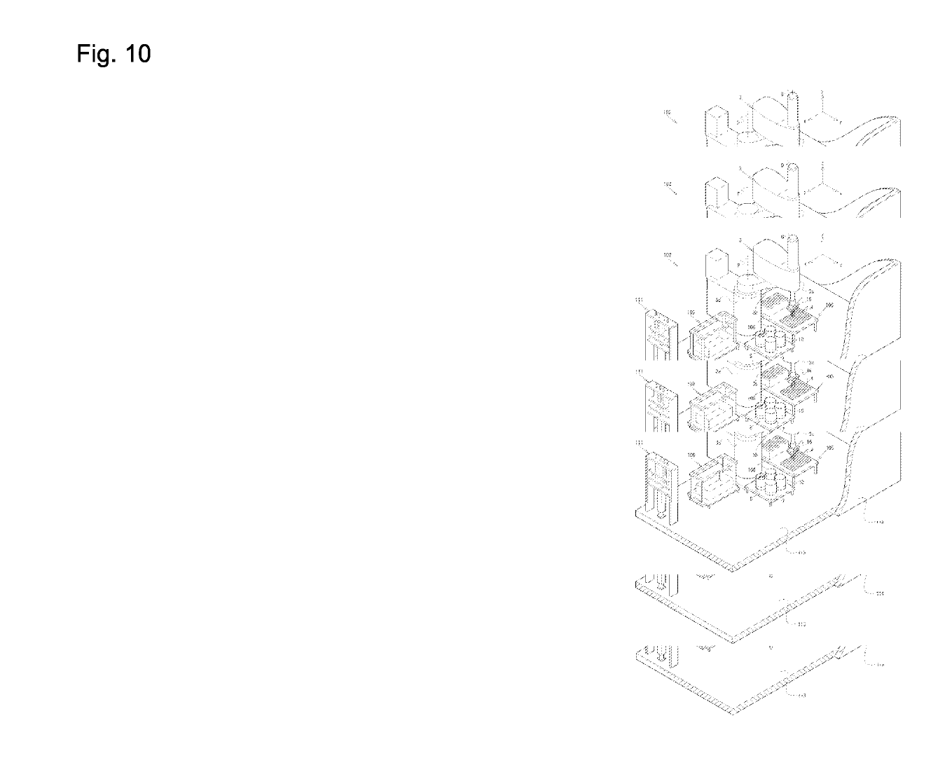

### BRIEF DESCRIPTION OF THE DRAWINGS

[FIG. 1] A perspective view schematically showing the configuration of a culture system of the present invention.

[FIG. 2] A perspective view of an antifoam device looking from an angle, which prevents foam generation by removing droplet from the end of the nozzle.

[FIG. 3] A cross-sectional view of a drainer unit installed in the antifoam device, cut lengthwise along the disposed direction of the liquid-removing apertures.

[FIG. 4] A perspective view of a drainer unit having a grill, looking from an angle.

[FIG. 5] A cross-sectional view of a drainer unit provided with drainers under the liquid-removing apertures, cut lengthwise along the disposed direction of the liquid-removing apertures.

[FIG. 6] A perspective view showing a partially enlarged nozzle unit of a handling mechanism installed in the culture system.

[FIG. 7] A perspective view of a mold (first container) placed inside a second container, shown by partially cutting away the partition wall of the second container.

[FIG. 8] A cross-sectional view of a funnel, a mold and a second container, cut lengthwise along the mold.

[FIG. 9] A cross-sectional view of an enlarged mold and a filter placed inside the mold, cut lengthwise.

[FIG. 10] A partially exploded perspective view schematically showing the internal configuration of a culture system, shown by partially cutting away a part of the cover of the culture system.

### EMBODIMENTS FOR CARRYING OUT THE INVENTION

The present inventor has developed a method for producing a three-dimensional cell construct made only of cells, the method comprising: placing a cell mass in a chamber having micropores that allow a culture fluid to pass therethrough, where the culture fluid is contained in the chamber such that a part of the cell mass makes contact with the gas phase; and culturing the cell mass in a culture fluid that of an excessive amount as compared to that of the culture fluid in the chamber (U.S. Pat. No. 4,122,280). This method, however, was conventionally conducted manually, and thus there were risks such as contamination (bacterial or mold contamination) or specimen mix-up. In the meantime, in order to keep the cost of the equipment investment low, instrumentation and automation have been needed. Thus, the present inventor has developed a robot for producing a three-dimensional construct to realize the above-described patented invention.

This robot handles a cellular construct in a solution by pneumatically drawing and discharging the solution with a disposable tip.

However, since pneumatical drawing and discharge of the solution causes air to enter and exit the tip, a droplet or foam remains at the tip end. Therefore, after a set of cells are transferred, a bubble is generated upon a stroke for handling the next set of cells. If this bubble gets into the container for preparing the three-dimensional cell construct, a cell mass may be trapped in this bubble, which inhibits the cell masses to associate with each other, making production of the three-dimensional construct difficult. If the bubble is physically ruptured, the culture fluid may disperse beyond the expected area and may result in contamination. Moreover, use of a surfactant such as a bubble eliminating agent may not be appropriate in terms of medical application. Furthermore, when the bubble is ruptured by heating, the cell may be exposed to heat damage and also associated with the risk of dispersion.

Therefore, according to the present invention, a drainer unit for easily removing the droplet or foam caused at the tip, more specifically, a foam removal device in an automatic cell handling robot, and a culture system using this device were developed. Specifically, when a nozzle or a tip attached to the end of the nozzle (unless otherwise indicated, referred to as “nozzle”) provided on handling means that is capable of drawing and discharging a culture fluid is used to transfer a cell from one container to the other, a droplet or foam is caused near the end of the nozzle upon this drawing or discharge. The present invention relates to liquid removal means for removing this droplet or foam from the nozzle, which serves as a drainer unit. The liquid removal means of the present invention is used in an apparatus (robot) for handling a cell. In one aspect, the present invention is characterized by having a plate-like form provided with an aperture having a size that allows the nozzle to pass up and down therethrough. By providing this aperture, the nozzle can pass up and down through the drainer unit. As the droplet or foam makes contact with the unit near the aperture upon passing, the droplet or foam runs along the drainer unit to come out from the nozzle, thereby removing the droplet or foam caused in the nozzle.

The drainer unit of the present invention, however, is not limited to the embodiment used with the above-described apparatus, and can be applied to cases where a droplet or foam attached to a pipette or a tip upon handling, i.e., drawing and discharging, a cell with the pipette or the tip, needs to be removed. In this case, the drainer unit can be provided, for example, at the opening of the culture container.

Furthermore, the present invention relates to a culture system that utilizes the above-described liquid removal means.

Specifically, a culture system of the present invention is characterized by comprising:
handling means for handling a cell mass;
a first container for reserving the cell mass supplied from the handling means and having micropores that allow communication of a culture fluid with the external area;
a second container for accommodating the first container, where a culture fluid fills around the first container in an excessive amount as compared to the amount of the culture fluid in the first container; and
liquid removal means for removing droplet or foam remaining in the handling means after supplying the cell mass into the first container.

The means for handling the cell mass is not limited as long as it is capable of manipulating the cell mass and it may be provided with, for example, a mechanism capable of suctioning and discharging a cell mass and a transfer control mechanism for spatially transferring this mechanism. It may also be provided with, as an alternative for the above-described mechanism for suctioning and discharging a cell mass, for example, a shovel mechanism for scooping and releasing a cell mass and a transfer control mechanism for spatially transferring the shovel mechanism.

An example of the above-described suction/discharge mechanism includes a nozzle, while an example of the transfer control mechanism includes a positioning device for transferring the suction/discharge mechanism in triaxial directions XYZ. As such a transfer control mechanism, for example, an industrial robot such as a horizontal articulated robot or a vertical articulated robot may be used. According to the present invention, a culture fluid can be suctioned/discharged directly with a nozzle provided in the system, and it is preferable to attach a disposable pipette tip to the nozzle.

The shape of the first container is not particularly limited as long as it is capable of receiving a cell mass and allows communication of the culture fluid with the external area. As means for allowing communication of the culture fluid with the external area, for example, a pore with a smaller diameter than that of the cell mass can be provided at the bottom or the side of the container. Alternatively, the bottom of a cylindrical container may be provided with a member having a plurality of micropores (as will be described in detail below).

Similar to the first container, the shape of the second container is also not particularly limited as long as it can accommodate a culture fluid that is excessive in the amount as compared to the amount of the culture fluid in the first container. A typical example of such second container includes a container shaped to surround the first container. The culture fluid can communicate between the first container and the second container, by which nutrient contents and the like contained in the culture fluid in the second container can be supplied to the cell mass in the first container.

The liquid removal means can be any means as long as it can remove the droplet or foam (hereinafter, unless otherwise indicated, referred to as “droplet”) remaining in the handling means after the cell mass is supplied into the first container. Such liquid removal means may employ, for example, a method in which a droplet attached to the handling means is removed by blowing, a method in which a droplet attached to the handling means is removed by aspiration (drawing), a method in which a droplet attached to the handling means is removed by gathering the droplet at an apex protruding downward in the direction of gravitational force to drip, or the like. Hereinafter, a culture system provided with the handling means, the first container, the second container and the liquid removal means described will be described. This is, however, merely an example and the present invention is not limited to the following embodiment.

FIG. 1 is a perspective view showing a general outline of a culture system of the present invention. As shown in FIG. 1, a culture system 2 is provided with a handling device 3, an incubator 7 as a second container, an antifoam device 8 and a medium reservoir 10.

The handling device 3 is provided with nozzles 20 described below (see FIG. 6), and a transfer control mechanism for controlling three-dimensional transfer of the nozzles 20. For example, tips 22 can be attached to the ends of the nozzles 20 as shown in FIG. 6. The transfer control mechanism can accurately control the transfer of the nozzles 20, for example, in the horizontal directions (XY-directions) and the vertical direction (Z-direction) so as to allow handling of the cell masses. The cell masses handled by the nozzles 20 can be accommodated, for example, in wells of a well plate 4 where the nozzles 20 suction the cell masses from these wells and discharge into a mold 27 as a first container placed in a second container (see FIG. 7) so that the cell masses are accommodated in the mold 27.

An antifoam device 8 is placed adjacent to an incubator 7, where the antifoam device 8 prevents bubble generation upon a stroke, and thus prevents bubble generation in the first container, by removing droplets from the handling device 3. Since the handling device is provided with the nozzles 20, the antifoam device 8 is provided with a mechanism for removing droplets from these nozzles 20.

FIG. 2 is a perspective view of a drainer unit for preventing bubble generation upon a stroke by removing droplets from the nozzle ends and an antifoam device comprising this drainer unit, looking from an angle.

As shown in FIG. 2, the antifoam device 8 is provided with the drainer unit 40. For example, four liquid-removing apertures 40a are formed in the drainer unit 40 according to the layout of the nozzles 20, and each of the liquid-removing apertures 40a has an aperture size specified to be capable of removing a droplet or foam remaining at the end of the tip 22 attached to the nozzle 20. The number of these liquid-removing apertures 40a may appropriately be changed according to the number of the nozzles 20. The drainer unit 40 is arranged so as to be surrounded by a partition wall 42 which reduces dispersion of the droplets removed from the ends of the tips 22 by the drainer unit 40.

An embodiment of the drainer unit may, for example, be a drainer unit having liquid-removing apertures with a predetermined clearance with respect to the outer diameters of the nozzle ends as shown in FIG. 3 or a drainer unit having a grill as shown in FIG. 4. Since these liquid-removing apertures 40a of the drainer unit have an aperture diameter that allows the whole or a part of the nozzle to pass therethrough, the droplets at the nozzle ends run to the drainer unit as the nozzle ends approach the liquid-removing apertures of the drainer unit, thereby removing the droplets from the nozzle ends. A mechanism of removing droplets from the ends of the nozzles 20 will be described below.

FIG. 3 is a cross-sectional view of a drainer unit provided in the antifoam device, cut lengthwise along the disposed direction of the liquid-removing apertures, and an embodiment in which tips are attached at the ends of the nozzles will be described.

As shown in FIG. 3(A), liquid-removing apertures 40a are formed in the drainer unit 40 for removing droplets from the ends of the tips 22. The apertures are formed to have a diameter that gives a predetermined clearance with respect to the outer diameters of the ends of the tips 22 (distance between the drainer unit and the tip end). This clearance allows only the droplets 47 gathered at the ends of the tips 22 to move to the drainer unit 40 without the ends of the tips 22 touching the drainer unit 40. Depending on the amount of the liquid and the surface tension, the clearance may be 10.0 mm or less, 9.0 mm or less, 8.0 mm or less, 7.0 mm or less, 6.0 mm or less, 5.0 mm or less, 4.0 mm or less, 3.0 mm or less, 2.0 mm or less or 1.0 mm or less, preferably 1.0 mm or less and more preferably 0.5 mm or less. By placing the ends of the tips 22 to be spaced apart from the liquid-removing apertures with such a clearance, droplet can be removed from the ends of the tips 22 as shown in FIG. 3(B). Accordingly, bubble generation can be prevented upon discharging the next liquid into a funnel 28 of an incubator 7. The droplets that moved to the drainer unit 40 are collected in the antifoam device 8. The tips may make contact with the liquid-removing apertures to remove the droplets from the tip end as long as the ends of the tips are not worn away, in which case more accurate transfer control of the tips is required.

According to the present invention, an antifoam device 8 can be provided exclusively as a drainer unit or a drainer unit can be provided in the second container so that the second container can also serve as the antifoam device. For example, similar to the embodiment of the antifoam device shown in FIG. 2, when a drainer unit is arranged in the upper part of the second container (preferably, near the opening of the incubator) such that the first container and the drainer unit are coaxially arranged in the vertical direction (coaxial in the Z-direction), the tips 22 accommodating cell masses pass through the liquid-removing apertures of the drainer unit and descend to a culture mold in the container 1 and ascend after discharging the cell masses to again pass through the liquid-removing apertures of the drainer unit. The droplets attached to the tips make contact with the drainer unit near the liquid-removing apertures upon this passing, whereby the droplets are removed from the tips.

The order of draining may be such that the draining is conducted before, after or both before and after discharging the cell masses into the first container.

The apertures are formed to have a diameter such that the whole tips can pass therethrough and that they are spaced with a predetermined clearance with respect to the outer diameters of the ends of the tips 22. This clearance allows only the droplets 47 gathered at the ends of the tips 22 to move to the drainer unit 40 without the ends of the tips 22 touching the drainer unit 40. In this manner, the cells can be discharged into the mold and the droplets can be removed, by a single stroke. In the case of this embodiment, the removed droplets directly run along the unit and drip into the culture bath. There is no influence on the subsequent stroke since the droplets would be mixed with the culture fluid in the first container and the foam would stay or vanish on the surface of the culture fluid in the first container. Of course, there is no problem of contamination.

Removal of droplets 47 from the ends of the tips 22 with the drainer unit 40 can suppress bubble generation upon actuation of the nozzles 20. Once bubbles are generated, the bubbles may stay at a converging part 28a of the funnel 28 (see FIG. 7) or the communication opening between the funnel 28 and the mold 27 may be blocked, in which case it becomes difficult to accommodate the cell masses in the mold 27. Use of the drainer unit 40, however, can prevent bubble generation and suppress trapping of the cell masses in the bubbles, thereby maintaining efficient introduction of the cell masses.

FIG. 4 is a perspective view for illustrating a drainer unit provided with grills (mesh parts). The shape of the drainer grill is not particularly limited and may appropriately be determined, for example, according to the shape or the size of the tip.

In a drainer unit 50 illustrated in FIG. 4(A), droplets can be removed from the ends of the tips 22 by accurately controlling the descending motion of the tips 22. The drainer unit 50 is provided with grills (mesh parts) 52 and the ends of the tips 22 are controlled to descend so as to leave a predetermined space with respect to these grills 52. The spaces between the ends of the descended tips 22 and the grills 52 may be, as described before, 10.0 mm or less, 9.0 mm or less, 8.0 mm or less, 7.0 mm or less, 6.0 mm or less, 5.0 mm or less, 4.0 mm or less, 3.0 mm or less, 2.0 mm or less or 1.0 mm or less, preferably 1.0 mm or less and more preferably 0.5 mm or less. By placing the ends of the tips 22 to face the grills 52 with such a space, the droplets can move from the ends of the tips 22 to the drainer unit 50, thereby removing the droplets from the ends of the tips 22. In addition, as shown in FIG. 4(B), the drainer unit 54 can be provided with grills 56 having finer grids. This drainer unit 54 can also be used to remove the droplets from the ends of the tips 22. In an embodiment where a drainer unit is placed in the upper part of the container 1, droplets at the ends of the tips 22 can be removed by providing apertures that allow the tips 22 to pass therethrough and controlling the clearance between the tips and the wall surfaces defining the apertures.

Additionally, although the drainer unit 40 illustrated in FIG. 3 is only provided with liquid-removing apertures 40a having a predetermined clearance with respect to the outer diameters of the ends of the tips 22, drainers can be provided under the liquid-removing apertures to remove the droplets by flexibly making contact with the tip ends.

FIG. 5 is a cross-sectional view of a drainer unit provided in an antifoam device, cut lengthwise along the disposed direction of the liquid-removing apertures. As shown in FIG. 5(A), drainers 65 are provided on the back of the drainer unit 60, which come closer to the outer diameters of the ends of the tips 22 inserted into the liquid-removing apertures 60a. The drainers 65 are provided with narrowed parts 65a that narrow inward, which can come close to the outer peripheries of the ends of the tips 22.

The drainers 65 are formed of a flexible elastic material, for example, a polymer such as polyethylene or polypropylene. By using such a highly flexible elastic material, the drainers 65 will be inwardly energized and thus capable of receiving the ends of the tips 22 while making contact with the outer peripheries thereof.

When the narrowed parts 65a of the drainers 65 make contact with the outer peripheries of the ends of the tips 22, the droplet at the ends of the tips 22 move to the drainers 65 and gradually accumulate at the lower ends of the drainers 65. As the ends of the tips 22 are inserted into the liquid-removing apertures 60a, the ends of the tips 22 make contact with the narrowed parts 65a of the drainers 65. The narrowed parts 65a are flexible and pushed out by the outer peripheries of the ends of the tips 22 as the ends of the tips 22 are further inserted downwardly. Through such a series of movements of the ends of the tips 22 with the drainers 65, the droplets attached to the ends of the tips 22 move to the ends 65b of the drainers 65 via the narrowed parts 65a. By providing the drainers 65, a liquid having a higher viscosity than that of water or the like can also be removed from the ends of the tips 22, thereby preventing bubble generation upon discharging the subsequent liquid into the funnel 28 of the incubator 7. In the embodiments illustrated in FIGS. 3 and 4, the ends of the tips 22 need to be accurately controlled so that they do not make contact with the drainer units 40 and 50, the flexible drainers 65 as shown in FIG. 5 allow convenient descent control of the tips 22 to remove the droplets from the ends of the tips 22. Furthermore, the drainer unit shown in FIG. 5 can also be arranged in the upper part of the incubator 7 to remove droplets as described above.

The shape of the drainers is not limited to that shown in FIG. 5(A) and may take, for example, a shape shown in FIG. 5(B). The drainers 70 shown in FIG. 5(B) are formed to have a shape whose cross-section gently curves inward and is formed of a flexible elastic material. As the tips 22 are inserted into liquid-removing apertures 68a of a drainer unit 68, the ends 70a of the drainers 70 make contact with the outer peripheries of the ends of the tips 22, and the droplets attached to the outer peripheries of the ends of the tips 22 are wiped away by the ends 70a of the drainers 70 as the tips 22 are pulled out from the liquid-removing apertures 68a, thereby removing the droplets from the ends of the tips 22. Thus, the droplets at the ends of the tips 22 can also be removed by using drainers having such a shape.

Hereinafter, each of the means in a cell culture system that employs liquid removal means of the present invention will be described.

The handling mechanism 3 (see FIG. 1) is a SCARA robot that has, for example, a main mechanism body 3a, a connecting arm 3b whose one end is axially and pivotally connected to the main mechanism body 3a and a movable body 3c connected to the other end of the connecting arm 3b. Since one end of the connecting arm 3b is attached to the main mechanism body 3a and the other end to the movable body 3c, the movable body 3c can pivot with respect to the rotation axis P. The movable body 3c is provided with a nozzle unit 16 that can move up and down, where the nozzle unit 16 can be positioned by freely moving in the XYZ-directions by the movement of the movable body 3c in the horizontal direction and the ascending/descending movement of itself.

FIG. 6 is a partially enlarged perspective view showing a nozzle unit 16 of the handling mechanism. As shown in FIG. 6, the handling mechanism 3 has nozzles 20 and can handle cell masses accommodated in wells 4a on a well plate via these nozzles 20. The nozzles 20 are arranged downwardly in the direction of gravity and droplets attached to the outer peripheries are supposed to flow down to the ends.

Since the movable body 3c is provided with a nozzle unit 16 which, in turn, is provided with the nozzles 20 for drawing and discharging a liquid, cell masses in any of the wells 4a on the well plate 4 can be handled. The number of nozzles is not particularly limited and it can appropriately be determined according to the number of the wells 4a, such as 1, 2, 4, 6, 8 or 16. In FIG. 6, a typical example of a system is illustrated in which the nozzle unit 16 has four nozzles 20 which can be provided with tips 22.

Although the nozzles 20 can draw and discharge cells by themselves, detachable tips 22 may be attached thereto, by which droplets attached to the outer periphery of the tips 22 flow down to the ends and accumulate at the end. The tips 22 are formed, for example, of a plastic material such as polyethylene or polypropylene, and detached in a tip box 109 after a predetermined routine. After detachment of the used tips 22, the movable body 3c moves above the mounted tip box 106 so that new tips 22 are attached to the nozzles 20.

The handling mechanism 3 is connected to a pump mechanism as represented by an electric cylinder or the like, by which the nozzles 20 can draw/discharge cell masses according to the depressurization/pressurization resulting from the pump mechanism. In this embodiment, the nozzles 20 are prevented from getting contaminated owing to the tips 22, an apparatus configuration can be employed in which a nozzle washing mechanism is provided instead of the tips.

The movable body 3c is provided with a translation control mechanism for drive controlling the nozzle unit 16 in the XYZ-directions, and a rotation control mechanism for drive controlling the rotation of the nozzle unit 16 with respect to the rotation axis Q. Accordingly, the nozzle unit 16 can move freely above the base 113 by the rotation of the movable body 3c with respect to the rotation axis P as well as the translation movement and the pivot movement that differs from this rotation of the movable body 3c.

The movement of the movable body 3c is precisely controlled, for example, by a motor such as a stepping motor or a servomotor and a motor controller for that motor.

A pump (cylinder) for pressurization/depressurization is connected to each of the nozzles 20 of the nozzle unit 16 and the amount of each of the nozzles 20 to draw or discharge is accurately controlled by precisely operating the pump.

Although the well plate 4 may have various numbers of wells 4a, as one example, a total of 96 wells in 8 rows and 12 columns are shown.

The movable body 3c is aligned by moving parallel to the surface of the well plate 4. Once the movable body 3c is aligned, the nozzle unit 16 descends from upper to lower part of the well plate 4. As the nozzle unit 16 descends, the ends of the tips 22 attached to the ends of the four nozzles 20 approach the wells 4a so that the nozzles 20 can draw the cellular suspension containing the cell masses in the wells 4a directly or via the tips 22.

FIG. 7 is a perspective view of a first container (a mold 27) placed inside an incubator 7 as a second container, shown by partially cutting away the partition wall of the second container. As can be appreciated from FIG. 7, the incubator 7 is provided with the mold 27, a funnel 28, a support 29 and the partition wall 31. The support 29 has a securing hollow 29a that is formed for detachably supporting the mold 27 (see FIG. 8). The mold 27 is secured by being fitted in this securing hollow 29a.

FIG. 8 is a cross-sectional view of the funnel, the mold and the second container (cross-section of components other than the cell mass), cut lengthwise along the mold. As can be appreciated from FIG. 8, a converging part 28a having a mortar-like slope is formed inside the funnel 28 which, in turn, is placed and fitted in the upper part of the mold 27. In this manner, the converging part 28a of the funnel 28 is in communication with the inside area of the mold 27.

In FIGS. 8 and 9, the bottom of the mold 27 is provided with a member (filter) having multiple micropores with a diameter smaller than that of the cell mass. These micropores allow the culture fluid to communicate with the external area of the mold while leaving the cell mass in the mold 27.

The inner area of the mold 27 communicates with the incubator 7 via a flow path 29c (FIG. 8). The incubator 7 is formed to retain an excessive amount of liquid. The volume of this incubator 7 can appropriately be determined according to the volume of the mold 27.

For example, the incubator 7 is provided to surround the mold 27 which can accommodate a culture fluid 25 in an excessive amount as compared to an amount of a culture fluid 24 accommodated in the mold 27. The culture fluid 25 in this incubator 7 communicates with the inner area of the mold via the micropores of the filter formed at the bottom of the mold 27, resulting in the culture fluid 24 in the mold. Thus, cell masses 23 in the first container can be cultured.

The culture fluid in the incubator 7 can communicate with the inner area of the mold 27 from the funnel side or the filter side and the cell masses in the mold 27 can be cultured with this culture fluid. With the excessive amount of culture fluid filling around the mold 27 and the cell masses reserved in the mold part 27b, a three-dimensional construct can be formed in an amount adequate for the treatment.

The amount of culture fluid fed into the incubator 7 is not particularly limited as long as the cells can proliferate/differentiate. For example, when a cell mass with a diameter of 4 mm and a thickness of 5 mm is to be cultured in the mold part 27b, the amount of culture fluid required in the incubator 7, for example, is 10-20 ml and the amount of the culture fluid fed into the incubator 7 needs to be about 5-10 times the volume of the mold part 27b.

The converging part 28a formed is open outward so as to accommodate cell masses discharged from the ends of, for example, four tips 22, and the cell masses discharged into the converging part 28a flow down the converging part 28a and enter the mold 27. In this manner, the cell masses can efficiently be introduced into the mold 27 via the funnel 28.

The mold 27, the funnel 28 and the support 29 are surrounded by the partition wall 31, by which addition of a foreign matter from the surrounding environment and dispersion of the culture fluid 33 to the surrounding environment is reduced/prevented.

FIG. 9 is an enlarged cross-sectional view of a mold and a filter placed inside the mold, cut lengthwise. As shown in FIG. 9, the mold 27 is configured from a main mold body 35 and a filter 37 incorporated in this main mold body 35.

The mold 27 forms a three-dimensional cell construct (cell plug) based on the cell masses such as spheroids that flow into the mold 27 via the funnel 28. In a preferred embodiment of the present invention, cells accommodated in the mold 27 float at the boundary between the culture fluid and the gas phase due to the surface tension. When the subsequent set of cell masses flow in, the uppermost cells that just flowed in, in turn, configure the boundary between the culture fluid and the gas phase. By repeating this while culturing the cell masses with an excessive amount of culture fluid filling the incubator 7, a three-dimensional cell construct can be formed. Furthermore, the mold part 27b sandwiched between the opening 27a and the filter 37 is formed in the mold 27, and a three-dimensional construct of any shape can be prepared by changing the shape of this mold part 27b.

At the bottom of the main mold body 35, a notch 35a is formed for a culture fluid flowing down from the mold part 27b to communicate with the external area of the mold 27 or for a culture fluid surrounding the mold 27 to communicate with the inner area of the mold 27. In this manner, the culture fluid can freely communicate, and cell masses in the mold part 27b can be cultured with an excessive amount of culture fluid filling around the mold 27. The cultivation can be conducted, for example, under the conditions at a temperature of 37° C. and a carbon dioxide concentration of 5%.

Examples of the material of the mold 27 include synthetic resins such as polystyrene, polyethylene, polypropylene, polycarbonate, polyamide, polyacetal, polyester, polyurethane and polyvinyl, silicon resins, synthetic rubbers, natural rubbers, ceramics and metal materials such as stainless.

The filter 37 is provided with micropores 37a for filtering the culture fluid from the cellular suspension so that the culture fluid that passes through these micropores 37a further flows down to the lower part of the main mold body 35 and enters the receiving part 29b formed in the support 29. The pore diameter of the micropores 37a is, for example, 10-500 μm so that the cell masses in the mold 27b do not pass toward the lower side of the filter 37 and only the culture fluid can go in and out via the filter 37.

A structural material of a filter is not limited as long as it is porous, and examples include a semipermeable membrane, a foamed or porous polymeric material, a sintered body, a porous glass and ceramics as well as naturally-derived polymeric substances with a porous structure such as chitosan, cellulose and dextran.

As a polymeric foam or porous material, materials, for example, polyolefin series such as polyethylene or polypropylene; diene series such as butadiene or isoprene; polyurethane; vinyl polymers such as polyvinyl chloride, acrylamide, polystyrene or polyvinyl alcohol; condensates such as polyether, polyester, polycarbonate or nylon; or silicon or a fluorine resin can be applied.

Cells that can be targeted by a culture system of the present invention are, for example, undifferentiated or differentiated cells of a stem cell (ES cell, umbilical blood-derived cell, undifferentiated mesenchymal cell, etc.), a somatic cell, a tumor cell or the like.

A fibroblast cell, a stem cell, a vascular endothelial cell, an epidermal cell, an epithelial cell, an osteoblast, a chondrocytic cell and an adipose cell can easily be induced to differentiate from an undifferentiated mesenchymal stem cell. Cells such as articular chondrocytic cells and osteocytes can also be used.

Thus, the present invention can be applied to an articular cartilage, a bone as well as an adipose cell such as breast, a ligament and the like, while using a mesodermal tissue as a core.

Cells are broadly grouped into anchorage-independent cells and anchorage-dependent cells, where blood cells and immune system cells belong to the former while epidermal cells and osteocytes belong to the latter. The epidermal cells and osteocytes will die in floating conditions in a culture fluid and need to be proliferated by adhering them to a Schale such as glass. Therefore, when the cells are made to gather at the same place in polytetrafluoroethylene, the cells will adhere to each other seeking for anchorage, thereby resulting in forming a cellular aggregate, namely, a spheroid. Furthermore, when the spheroids adhere and fuse together, a larger shape will result.

Due to intervention of spheroids, the cells enter the stationary phase of the cell cycle, whereby production of a protein is considered to increase. Thus, according to the present invention, since the cells are induced to enter the stationary phase, they are preferably once made into spheroids and then formed into a predetermined shape.

The culture fluid used for cell cultivation may be commonly used synthetic or natural medium depending on the cell to be cultured.

Considering infection with a bacterial virus or the like resulting from an animal-derived substance, supplying season and quality stability, a synthetic medium is favorable.

As a synthetic medium, for example, α-MEM (Minimum Essential Medium), DMEM (Dulbecco's modified Eagle medium) RPMI 1640 medium, CMRC medium, HAM medium, DME/F12 medium, MCDB medium or the like can be used.

These media may appropriately be added with a proliferative factor, a growth factor, a biologically active substance such as a hormone, or other various substances having pharmacological action. Addition of such substances can give or change the function of the medium.

Examples of a growth factor or a cellular proliferative factor include bone morphogenetic protein (BMP), fibroblast growth factor (FGF), transforming growth factor-β (TGF-β), insulin-like growth factor (IGF), platelet derived growth factor (PDGF), vascular endothelial growth factor (VEGF), known serum components such as transferrin (concentration is adjusted appropriately), various vitamins and antibiotics such as streptomycin.

Examples of hormones include insulin, transferrin, dexamethasone, hydrocortisone, thyroxine, 3,3′,5-triiodothyronine, 1-methyl-3-butylxanthine and progesterone.

Typically, examples of other biologically active substances include vitamins such as ascorbic acid (particularly, L-ascorbic acid), biotin, calcium pantothenate, ascorbic acid 2-phosphate and vitamin D, proteins such as serum albumin and transferrin, lipids, fatty acid sources, linoleic acid, cholesterol, pyruvic acid, DNA and RNA synthetic nucleoside, glucocorticoid, retinoic acid, β-glycerophosphate and monothioglycerol.

The cultivation temperature of the cells is typically 35-40° C. and preferably around 37° C. The cultivation period may appropriately be adjusted according to the size of a cell mass of interest. For example, in order to culture embryonic stem cells, it is generally well known to conduct the cultivation, for example, under the conditions at a temperature of 37° C. and a carbon dioxide concentration of 5%.

Spheroids derived from the embryonic stem cells can be formed under the above-described conditions. Hereinafter, an embodiment of the present invention will be described in more detail.

Hereinafter, more specific configuration of the above-described culture system will be described. FIG. 10 is a partially exploded perspective view schematically showing the internal configuration of the culture system, shown by partially cutting away a part of the cover of the culture system of the present invention. Like reference numerals designate like parts of FIGS. 1-9 and the explanation thereof is omitted.

As shown in FIG. 10, the culture system 102 is provided with the handling mechanism 3 as handling means, a stage board 105 on which the well plate 4 is set, the mounted tip box 106, the incubator 7, the antifoam device 8, the detached tip box 109 for used tips, a medium reservoir 10, an electric cylinder 111, a temperature controller (not shown), an aeration/deaeration mechanism and the like. The handling mechanism 3, the well plate 4, the stage board 105, the incubator 7, the tip box 109 and the electric cylinder 111 are each secured on a base 113 and covered with a cover 114. Alternatively, the whole system may be covered with cover 114, by which grit and dust from outside can be prevented from entering inside the apparatus.

The handling mechanism 3 is arranged at the center of the base 113 while the movable body 3c of the handling mechanism 3 can rotatably move around axis P. As the handling mechanism 3, for example, a robot such as a horizontal articulated robot or a vertical articulated robot can be used.

The handling mechanism 3 has the main mechanism body 3a secured to the base 113, the connecting arm 3b whose one end is pivotally and axially connected to the main mechanism body 3a and the movable body 3c connected to the other end of the connecting arm 3b. Since one end of the connecting arm 3b is attached to the main mechanism body 3a and the other end to the movable body 3c, the movable body 3c can pivot with respect to the rotation axis P.

The stage board 105 provided with the mounted tip box 106, on which a well plate 4 can freely be set, the incubator 7, the antifoam device 8, the medium reservoir 10 and the detached tip box 109 are arranged around the handling mechanism 3. Although FIG. 10 shows an embodiment in which the antifoam device 8 having the drainer unit and the incubator 7 are separately arranged, the incubator 7 may alternatively be provided with the drainer unit.

The mounted tip box 106 comprises a plurality of unused tips that are to be attached to the nozzle 20 (see FIG. 6), by which the tips can be exchanged for every culture operation. The well plate 4 accommodates, for example, cultured cell masses (spheroids), and this cell masses accommodated in the well plate 4 are used to form a three-dimensional construct. The medium reservoir 10 accommodates a medium that is injected into the incubator 7 upon forming a three-dimensional cell construct. The detached tip box 109 is used to accommodate tips that have been used and detached from the nozzles.

Next, an exemplary operation of the present invention will be described by using the system illustrated in FIG. 10.

The well plate 4 accommodating cell masses such as spheroids is set on the stage board 105, and the movable body 3c of the handling mechanism 3 moves above the well plate 4, whereby the nozzle unit 16 descends. As the nozzle unit 16 descends, each of the tips 22 attached to the four nozzles 20 enters the corresponding well 4a, and the cell mass accommodated by each well 4a is drawn into each tip 22.

After the cell mass is drawn into each tip 22, the movable body 3c moves above the incubator 7 and the nozzle unit 16 descends toward the funnel 28. Once the nozzle unit 16 descends, the cell masses are discharged from the tips 22 into the converging part 28a of the funnel 28. Since the cell masses discharged into the converging part 28a are heavier than the solution, they run down the converging part 28a and are received by the mold part 27b of the mold 27.

Once the cell masses are discharged, where appropriate, the movable body 3c of the handling mechanism 3 may move above the medium reservoir 10 and the nozzle unit 16 may descend to draw the medium from the medium reservoir 10 into the tips 22. After the medium is drawn, the movable body 3c moves above the incubator 7 and the medium in the tips 22 is discharged from the side of the funnel 28 into the support 29 of the incubator 7 so that an excessive amount of medium fills around the mold 27.

The cultivation conditions in this case are, for example, at a temperature of 37° C. and a carbon dioxide atmosphere of 5%. After a lapse of a predetermined cultivation period, a three-dimensional construct in an amount adequate for treatment is formed in the mold part 27b of the mold 27. The droplets at the ends of the tips 22 can be removed with the drainer unit 40 installed in the antifoam device 8.

Following formation of the three-dimensional construct, the funnel 28 mounted on the mold 27 is detached and further the mold 27 is detached from the support 29 so that the three-dimensional cell construct can be collected from the mold 27.

Accordingly, a culture system of the present invention further comprises a drainer unit in an antifoam device or an incubator so as to remove droplets remaining at the ends of the tips while preventing bubble generation and reducing dispersion of the droplets to the surrounding area. Moreover, according to the present invention, cell masses can be poured into a mold to form a three-dimensional cell construct used for regenerative treatment without manual operation so as to reduce bacterial or mold contamination and enhance convenience. In addition, by using a SCARA robot as the handling mechanism, a compact culture system that requires smaller installation space can be realized.

### DESCRIPTION OF REFERENCE NUMERALS

2 Culture system
3 Handling mechanism (handling means)
4 Well plate
4a Well
7 Incubator (second container)
8 Antifoam device
16 Nozzle unit
20 Nozzle
22 Tip
23 Cell mass
24 Culture fluid
25 Culture fluid
27 Mold (first container)
28 Funnel
29 Support
35 Main mold body
37 Filter
37a Micropore
40 Drainer unit (liquid removal means)
40a Insertion aperture
45 Drainer
50 Drainer
102 Culture system
105 Stage board
109 Tip box
111 Electric cylinder

1-10. (canceled)

11. A system for culturing a cell mass comprising:
handling means for handling the cell mass;
a first container for reserving the cell mass supplied from the handling means, the first container having micropores that allow communication of a culture fluid with the external area;
a second container for accommodating the first container, where a culture fluid that fills around the first container is in an excessive amount as compared to the amount of the culture fluid in the first container; and
liquid removal means for removing a droplet or foam remaining in the handling means upon said handling.

12. The culture system according to claim 11, wherein the handling means comprises a nozzle capable of drawing in and discharging a culture fluid containing the cell mass, so as to handle the cell mass via the nozzle.

13. The culture system according to claim 12, wherein the liquid removal means comprises a drainer unit for removing a droplet or foam, which is caused upon the drawing or discharge, from the end of the nozzle when the end of the nozzle approaches the drainer unit.

14. The culture system according to any one of claims 11 to 13, wherein the first container is a mold for molding a three-dimensional cell construct by culturing the reserved cell masses, wherein a member (filter) having micropores is provided at the bottom of the mold.

15. The culture system according to claim 11, comprising a funnel that opens outward at the opening of the first container.

16. A drainer unit for removing a droplet or foam, which is caused upon drawing or discharge, near the end of the nozzle provided on the handling means that is capable of drawing and discharging a culture fluid, wherein the drainer unit is provided with an aperture having a size that allows the nozzle to pass up and down therethrough.

17. The unit according to claim 15, which removes the droplet or the foam from the end of the nozzle by making the droplet or the foam to make contact with the unit in the vicinity of the aperture.

18. The unit according to either one of claims 16 and 17, which is placed at the opening of the culture container.

19. The culture system according to claim 13, wherein the drainer unit is provided with a liquid-removing aperture having a size that allows the nozzle to pass up and down therethrough, and is coaxially arranged with the first container in the vertical direction.

20. The culture system according to claim 19, wherein the removed droplet or foam runs along the drainer unit and drips into the first container.
